(19) United States
(12) Patent Application Publication
(10) Pub. No.: US 2024/0038397 A1
(43) Pub. Date: Feb. 1, 2024

(54) METHODS AND APPARATUS FOR IDENTIFYING RISK OF POSTCARDIOTOMY CARDIOGENIC SHOCK IN PATIENTS

(71) Applicant: Abiomed, Inc., Danvers, MA (US)

(72) Inventors: Randi Parks, Danvers, MA (US); Eugene Blackstone, Danvers, MA (US); Edward Soltesz, Danvers, MA (US); Elise Jortberg, Danvers, MA (US)

(73) Assignee: Abiomed, Inc., Danvers, MA (US)

(21) Appl. No.: 18/355,630

(22) Filed: Jul. 20, 2023

Related U.S. Application Data
(60) Provisional application No. 63/392,414, filed on Jul. 26, 2022.
(60) Provisional application No. 63/444,122, filed on Feb. 8, 2023.

(51) Int. Cl. G16H 50/30; G16H 10/60; G16H 50/50; G06N 20/00

(52) U.S. Cl. CPC G16H 50/30; G16H 10/60; G16H 50/50; G06N 20/00

(57) ABSTRACT

Methods and systems for predicting whether a patient is likely to develop post-cardiotomy cardiogenic shock (PCCS) are described. The method includes receiving medical information for a patient, extracting one or more features from the received medical information, providing the one or more features as input to a trained classification model configured to output a risk assessment that the patient is likely to develop PCCS, and outputting an indication of the risk assessment.

### RELATED APPLICATIONS

This application claims the benefit under 35 U.S.C. § 119(e) to U.S. Provisional Patent Application No. 63/392,414, filed Jul. 26, 2022, and titled, “METHODS AND APPARATUS FOR IDENTIFYING RISK OF POSTCARDIOMTOMY SHOCK IN PATIENTS,” and claims the benefit under 35 U.S.C. § 119(e) to U.S. Provisional Patent Application No. 63/444,122, filed Feb. 8, 2023, and titled, “METHODS AND APPARATUS FOR IDENTIFYING RISK OF POSTCARDIOMTOMY SHOCK IN PATIENTS,” the entire contents of each of which is incorporated by reference herein.

### FIELD OF THE INVENTION

This disclosure relates to identifying patients at risk of developing postcardiotomy cardiogenic shock.

### BACKGROUND

Postcardiotomy cardiogenic shock (PCCS) is a relatively rare but a significant cause of death in patients who have undergone cardiac procedures. Although not consistently defined in the literature, PCCS relates to circulatory failure following cardiac surgery necessitating mechanical circulatory support (e.g., support with extracorporeal membrane oxygenation (ECMO) devices, ventricular assist devices (VADs), etc.) and/or administration of high-dose inotropes.

### SUMMARY

In some embodiments, a method of predicting whether a patient is likely to develop post-cardiotomy cardiogenic shock (PCCS) is provided. The method includes receiving medical information for a patient, extracting one or more features from the received medical information, providing the one or more features as input to a trained classification model configured to output a risk assessment that the patient is likely to develop PCCS, and outputting an indication of the risk assessment.

In one aspect, the medical information for the patient includes one or more of an electronic health record , a laboratory report, a medical procedure report, physician notes, and a medical imaging report. In another aspect, the medical information includes structured data and unstructured data. In another aspect, extracting one or more features comprises extracting from the unstructured data, at least some of the one or more features using natural language processing. In another aspect, the one or more features include left ventricle ejection fraction and/or total bilirubin level. In another aspect, the method further includes receiving data indicating whether the patient developed PCCS, and retraining the trained classification model based, at least in part, on the received data. In another aspect, the risk assessment includes a numerical value, and outputting an indication of the risk assessment comprises displaying the numerical value and/or information based on the numerical value on a user interface. In another aspect, the method further includes performing based on the numerical value, categorization of the patient into a risk group of a plurality of risk groups, and outputting the indication of the risk assessment comprises outputting an indication of the risk group for the patient. In another aspect, performing categorization of the patient into a risk group comprises determining whether the numerical value is above a threshold value, and classifying the high risk for PCCS when it is determined that the numerical value is above the threshold value. In another aspect, outputting an indication of the risk group for the patient comprises displaying on a user interface a color coded indication of the risk group.

In another aspect, the risk assessment is a categorization of the patient into a risk group of a plurality of risk groups, and outputting the indication of the risk assessment comprises outputting an indication of the risk group for the patient. In another aspect, the trained classification model includes a trained neural network. In another aspect, the trained classification model includes a trained random forest model. In another aspect, the trained classification model includes a trained multivariate regression model. In another aspect, the method further includes receiving additional medical information, and retraining the trained classification model based on the additional medical information. In another aspect, the additional medical information includes medical information for a plurality of patients at a medical facility on which cardiac surgery was performed. In another aspect, the method further includes providing a user interface configured to display values for the one or more features, receiving user input via the user interface to change one or more of the values for the one or more features, simulating a risk assessment that the patient is likely to develop PCCS based, at least in part, on the changed one or more values, to generate a simulated risk assessment, and displaying, on the user interface, the simulated risk assessment. In another aspect, outputting an indication of the risk assessment comprises outputting a cumulative score associated with the risk assessment.

In some embodiments, a method of training a risk model to predict whether a patient is likely to develop post-cardiotomy cardiogenic shock (PCCS) is provided. The method includes receiving a dataset of patient medical information, selecting, from the dataset of patient medical information, training data based on PCCS criteria and defined data fields, wherein the training data includes patient medical information for at least two risk groups of patients, training the risk model using the selected training data, and outputting the trained risk model.

In one aspect, the method further includes defining a plurality of PCCS criteria and generating the at least two risk groups of patients based on the PCCS criteria. In another aspect, the method further includes receiving input via a user interface regarding the data fields to define, and defining the data fields based, at least in part, on the received input. In another aspect, the method further includes validating the trained model using at least some patient medical information not used to train the model, and outputting the trained risk model comprises outputting the validated trained model. In another aspect, the method further includes receiving an indication to update the trained risk model, and retraining the risk model in response to receiving the indication to update the trained risk model. In another aspect, the risk model includes a neural network. In another aspect, the risk model includes a random forest model. In another aspect, the risk model includes a multivariate regression model. In another aspect, the method further includes receiving additional medical information, and retraining the trained risk model based on the additional medical information. In another aspect, the additional medical information includes medical information for a plurality of patients at a medical facility on which cardiac surgery was performed.

In some embodiments, a computer-implemented system for predicting whether a patient is likely to develop post-cardiotomy cardiogenic shock (PCCS) is provided. The system includes at least one hardware computer processor, and at least one non-transitory computer readable medium encoded with a plurality of instructions that, when processed by the at least one hardware computer processor perform a method. The method includes extracting one or more features from medical information for the patient, providing the one or more features as input to a trained classification model configured to output a risk assessment that the patient is likely to develop PCCS, and outputting an indication of the risk assessment.

In some embodiments, at least one non-transitory computer readable medium encoded with a plurality of instructions that, when processed by the at least one hardware computer processor perform a method is provided. The method includes extracting one or more features from medical information for the patient, providing the one or more features as input to a trained classification model configured to output a risk assessment that the patient is likely to develop PCCS, and outputting an indication of the risk assessment.

### BRIEF DESCRIPTION OF DRAWINGS

FIG. 1 illustrates a flowchart of a process for classifying a patient with regard to the patient's risk for developing postcardiotomy cardiogenic shock (PCCS) in accordance with some embodiments of the present technology.

FIG. 2 illustrates examples of predictors that may be used in some embodiments of the present technology to predict a patient's risk for developing PCCS.

FIG. 3 illustrates a flowchart of a process for training a model for assessing a risk that a patient will develop PCCS in accordance with some embodiments of the present technology.

### DETAILED DESCRIPTION

Certain patients undergoing cardiac surgery may have an increased probability of developing post-operative cardiogenic shock, also referred to as postcardiotomy cardiogenic shock (PCCS). Currently, it is unknown which patients are most susceptible, and surgeons and other healthcare providers are left to gestalt to anticipate the occurrence of PCCS or wait until it happens. Unfortunately, patients that develop PCCS are at significantly increased risk of mortality and morbidity.

The inventors have recognized the benefits of using a risk model (e.g., using a large sample size of cardiac surgery patients) to predict which patients are most likely to develop PCCS. The output of the risk model may enable a healthcare provider (e.g., a physician) to provide patients having a predicted higher risk of developing PCCS with appropriate preemptive cardiac support (e.g., use of a mechanical circulatory device). Such preemptive support may provide the patient with a decreased chance of cardiac complications and/or an increased chance of survival following surgery compared with conventional reactive approaches where cardiac support is not provided until the patient shows evidence of PCCS. A risk model as described herein may identify baseline factors for predicting PCCS, and may be trained to set appropriate weights for each factor. As will be appreciated, the risk model may be implemented as a multivariable model. Accordingly, embodiments disclosed herein relate to a method of determining a patient's risk of developing PCCS. In some embodiments, a calculator may be developed to allow physicians to input patient baseline information for certain disclosed factors and predict the risk that the patient may experience PCCS.

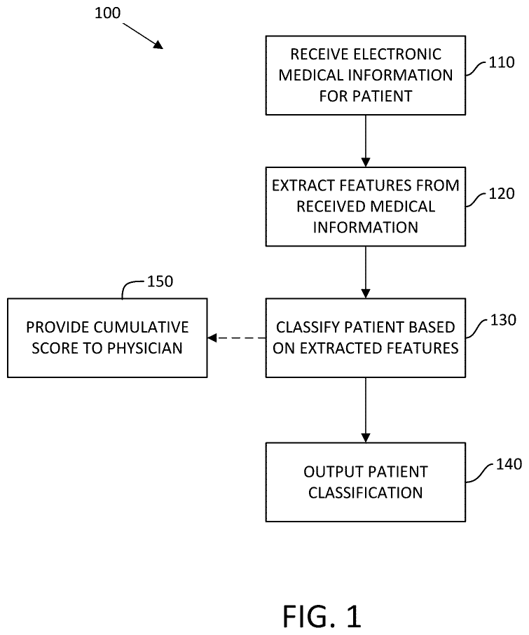

Turning now to the figures, FIG. 1 shows a flowchart of a process 100 for classifying a patient's risk for experiencing PCCS based on medical information, in accordance with some embodiments of the present technology. In act 110, electronic medical information for the patient may be received. The electronic medical information may include, but is not limited to, an electronic health record (EHR), laboratory reports, medical procedure (e.g., electrocardiogram) reports, physician notes, and medical imaging reports. In some embodiments, this information may be retrieved automatically from an electronic patient record. In other embodiments, act 110 may include receiving information that is entered into a patient calculator by a physician. In yet further embodiments, the received electronic medical information may include first information that is automatically retrieved from an electronic patient record and second information that is manually entered into a user interface (e.g., a user interface configured to display a patient calculator) by a physician or other healthcare provider.

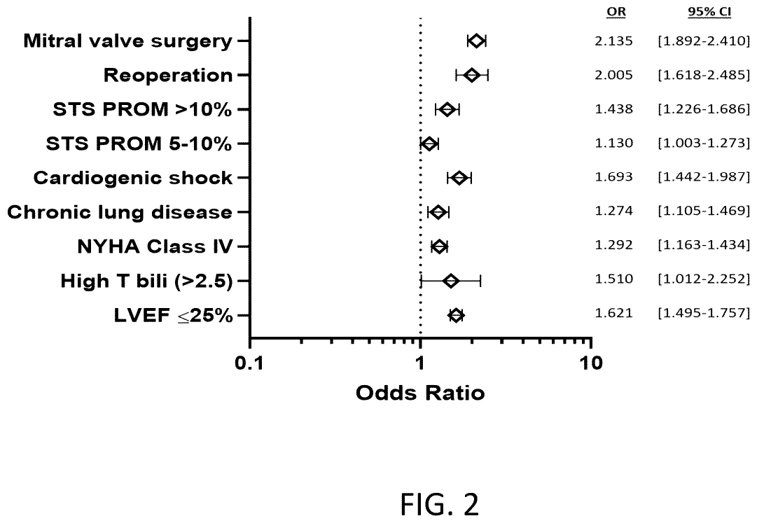

Process 100 may then proceed to act 120, where one or more features are extracted from the received medical information. The one or more extracted features may include, but are not limited to, heart function values, such as left ventricular ejection (LVEF), total bilirubin level, the existence of chronic lung disease, a New York Heart Association (NYHA) classification, the occurrence of cardiogenic shock, a Society of Thoracic Surgeons (STS) predicted risk score of mortality, whether the patient has undergone or will be undergoing mitral valve surgery, and/or whether a patient has undergone or will be undergoing a reoperation. FIG. 2 shows a chart of example features for predicting PCCS that may be used in some embodiments. Other features for predicting PCCS may additionally or alternatively be used in other embodiments.

As will be further appreciated, the received medical information may include structured data (e.g., organized based on an ontology, for example, using labels, fields, or other metadata associated with the data that can be used for feature extraction) and/or unstructured data (e.g., p hysicians' notes entered into a free text field in an electronic form).

As will be further appreciated, one or more features may be extracted using natural language processing (NLP) techniques. Such NLP techniques may be used to analyze text in the received medical information to infer information determined as features. In some instances, a first set of features is extracted using one or more manual techniques and a second set of features is extracted using automated (e.g., NLP) techniques.

Process 100 then proceeds to act 130, where the patient is classified (e.g., as being at high risk for developing PCCS) based, at least in part, on the extracted one or more features. The extracted feature(s) may be provided, for example, as input to a model (e.g., a trained machine learning model) trained to output a patient classification (also referred to herein as a “risk assessment”), or may be provided as input to an algorithm that weights the features to determine a score on which a classification for the patient is based. In some embodiments, values for two or more of the extracted features may be combined to create a derived feature that is not present in the received medical information. For instance, the values for two or more extracted features may be added, multiplied, subtracted, divided or combined in any other way to generate a value for the derived feature. Values for such derived features may be used to train the machine learning model and/or may be provided as input to the trained machine learning model in accordance with some embodiments. Examples of trained machine learning models include, but are not limited to, a neural network (e.g., a deep neural network), a logistic regression model, a random forest model, a Naive Bayesian model, and a decision tree model. Any single type of model or combinations of multiple types of models may be used as a risk model in accordance with the techniques described herein. In some embodiments, the trained machine learning model(s) may use ensemble methods, regression methods, or any other suitable methods for identifying the features and/or combinations of features that are most predictive (e.g., by associating them with greater weights) that a patient is likely to develop PCCS.

In some embodiments, the classification assigned to the patient may depend, at least in part, on the value of the extracted data. For example, patients having an LVEF of <=35%, such as an LVEF <=25% may have a higher risk of PCCS. Similarly, a patient with a Class III or Class IV NHYA rating may have a higher risk of PCCS. Worsening (e.g., higher) total bilirubin levels also may be indicative of a higher risk of PCCS. For instance, a total bilirubin level >2.5 may be indicative of a higher risk of PCCS. An STS predicted risk of mortality (STS PROM) of >=5%, or >=10% may also be indicative of a higher risk of PCCS. Additionally, a patient having undergone or undergoing mitral valve surgery may be indictive of a higher risk of PCCS for that patient.

In some embodiments, a cumulative score may be determined based upon the value of the extracted data. As shown in FIG. 1, in some embodiments, the cumulative score may be provided to the physician (see act 150) instead of or in addition to a patient classification (see act 140).

In some embodiments, the data associated with each of the features (e.g., categories) may be weighted evenly. In other embodiments, the data associated with each of the features may be weighted differently. For example, a feature with a greater likelihood of causing PCCS (e.g., a patient undergoing reoperation or a patient with a LVEF <=35% or <=25%) may be weighted higher than another feature (e.g., a feature with less odds of causing PCCS).

As should be appreciated, other thresholds and/or features may be used in some embodiments that employ an algorithm-based risk model in which the extracted features and/or derived features are considered in a discretized manner (e.g., by comparing values of the features to threshold values). In embodiments that employ a trained machine learning model (e.g., a multivariate linear regression model, a neural network-based model, etc.), the values of the extracted features and/or derived features may be considered in a continuous manner (e.g., by not requiring comparison to a threshold value). Additionally, the trained model itself may learn the predictive value of combinations of features when assessing whether patients are likely to develop PCCS rather than the combinations of features being explicitly defined as derived features provided as input to the model. In this way, the model may learn (e.g., by setting appropriate weights) that a combination of features that individually may not indicate that the patient is likely to develop PCCS may nevertheless be a good indicator that the patient is a high-risk (or low-risk, medium-risk or some other risk category) PCCS patient.

Process 100 may then proceed to act 140, where the patient classification determined in act 130 may be output. The patient classification may be output in any suitable way. For instance, when a trained machine learning model is used to determine the patient classification, the output of the model may include a numerical value, and outputting the patient classification may be performed by outputting an indication of the numerical value and/or a range of numerical values associated with the numerical value output from the model. For example, the numerical value and/or a range of numerical values may be displayed on a user interface of a computing device such that a healthcare provider reviewing the output may consider the risk assessment when making a decision about whether the patient would benefit from additional cardiac support to reduce the risk of the patient developing PCCS. In some embodiments, the output of the model may be provided as a percent probability that the patient will develop PCCS.

In some embodiments, the patient classification determined in act 130 may represent one of a plurality of risk categories (e.g., high-risk, medium-risk, low-risk) indicating an extent to which the patient is at risk for PCCS. The risk category associated with the patient may be output in any suitable way. For instance, a visual indicator (e.g., red, yellow, green) may be displayed (e.g., on a user interface, in the patient's electronic health record, etc.) to indicate a likelihood that the patient will exhibit PCCS. In some embodiments, both a visual indicator (e.g., red, yellow, green) and a numerical value, numerical range, or percentage may be output in act 140 to indicate the likelihood that a patient will exhibit PCCS. In some embodiments, process 100 may be performed for each of a plurality of patients in a medical facility, and a list of patients classified as being most likely to develop PCCS may be output in act 140.

As will be appreciated, process 100 may be repeated for the same patient over a desired period of time, such as while the patient is in the hospital. In such instances, a patient may have the same output at act 140. In other instances, the output at act 140 may differ over time depending upon the received medical information (at act 110) and the classification at act 130. Accordingly, a physician may proceed to treat the patient differently depending upon the output in act 140 over time.

Information relating to the output 140 may be provided to a physician and/or other healthcare provider in any suitable way. For instance, the healthcare provider may be provided with information in a user interface that provides an indication of the output of the risk model and/or describes how the output was determined based on the extracted features. In some embodiments, based on a review of the output 140 provided on a user interface, a user (e.g., the healthcare provider) may interact with the user interface to change one or more aspects of the risk model, and such changes may be reflected in output 140. For example, the physician may change one or more criteria to see if it changes the risk assessment score. In some embodiments, the physician may use information about the risk assessment score to order one or more additional tests, and the results of such tests may be taken into consideration when determining the patient classification and/or calculating a risk assessment score.

In some embodiments, patients initially classified as “borderline” or having a medium risk for PCCS may be reclassified after additional information is made available. In such instances, the classification may be updated after such additional information is made available, with the new classification being relayed to the hospital staff.

As described herein, for embodiments of the present technology that employ models or algorithms in which extracted features may be weighted to determine the patient classification, the models/algorithms may be updated (e.g., retrained or tuned). For example, the models/algorithms may be updated based on feedback about whether the classification provided by the model/algorithm agreed or disagreed with an assessment of a healthcare professional reviewing the patient's risk for PCCS.

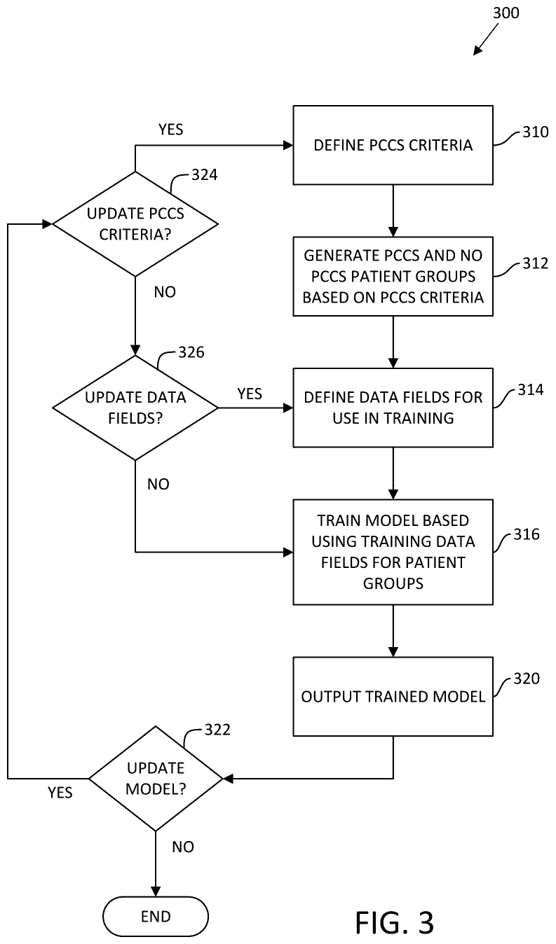

FIG. 3 illustrates a process 300 for training a model (e.g., a machine learning model) that may be used to assess risk of a patient developing PCCS in accordance with some embodiments of the present disclosure. Process 300 may begin in act 310, where PCCS criteria may be defined. As described herein, PCCS may not have a consistent definition in the scientific literature. As such, a patient's medical record may not explicitly identify whether the patient developed PCCS following cardiac surgery. Accordingly, as described herein, the inventors have recognized the benefit of creating criteria for reaching such a definition. In some embodiments, the PCCS criteria may include a definition of two classes — PCCS (positive class) and no PCCS (negative class). In other embodiments, more than two classes may be defined. For example, in addition to the PCCS and no PCCS classes defined above, the PCCS criteria may include a “Maybe PCCS” class and a “Maybe No PCCS” class. Including such classes may, for example, provide insight into how the model may perform for patients who may be characterized by a broader definition of PCCS than that used for the PCCS and no PCCS classes.

PCCS criteria defined in act 310 may be used to determine whether patients should be classified into one of a plurality of risk categories (e.g., low risk, medium risk, high risk) for developing PCCS. Process 300 may then proceed to act 312, where a plurality of patient groups are generated based on the PCCS criteria defined in act 310.

In the example shown in FIG. 3, two groups of patients—a PCCS patient group and a no PCCS patient group—are generated based on whether a patient in a dataset of patient medical data meets the PCCS criteria. For instance, a patient may be included in the PCCS patient group when the patient has any one of the following criteria: intra-aortic balloon pump (IABP) inserted post-surgery; catheter-based cardiac assist device inserted post-surgery; veno-arterial extracorporeal membrane (VA-ECMO) device inserted during surgery or post-surgery for cardiac failure or veno-venous extracorporeal membrane (VV-ECMO) device converted to VA-ECMO device during surgery or post-surgery; unplanned ventricular assist device (VAD) implanted in conjunction with cardiovascular surgery; or cardiac death or death during initial cardiac surgery. For instance, a patient may be included in the no PCCS patient group, if all of the following are true: patient was in intensive care unit for less than 10 days; no prolonged ventilation needed; no readmission to the hospital required; no end organ dysfunction detected; no open chest procedure needed; no cardiac assist device required; patient discharged from hospital alive; and no readmission to the intensive care unit required. It should be appreciated that the aforementioned PCCS criteria are merely exemplary and any suitable PCCS criteria may be used to define the patent groups used for training a risk model in accordance with the techniques described herein. It should also be appreciated that any suitable number of patient groups may be generated based on the PCCS criteria, and using two patient groups is merely one example. For instance, in some embodiments, the PCCS criteria may be used to generate three patient groups corresponding to the risk categories “low risk,” “medium risk,” and “high-risk,” and the model may be trained to identify patients in those three patient groups using the respective patient data as described herein.

Process 300 may then proceed to act 314, where a plurality of data fields may be defined for use as training data. For example, a dataset of patient medical data may include a large number (e.g., 100, 200, 300, 500) of data fields that could potentially be used for training a risk model. The inventors have recognized and appreciated that using a smaller number of data fields (i.e., less than all possible data fields) may be advantageous, as not all data fields may be as relevant to predicting risk of developing PCCS. The data fields may be defined in any suitable way. For example, a perturbation technique may be used to determine which data fields when included as input to the model contribute to producing an accurate risk assessment as reflected in the output of the risk model. Other techniques may alternatively be used. For example, one or more of fields with mostly empty data, fields for irrelevant features, fields for features used in defining the PCCS criteria, fields for post-intervention features, or fields that are highly correlated may not be used to train the risk model. In some embodiments, missing values in the data set may be imputed, the class ratio in the training data may be balanced, and/or the feature values for the defined data fields may be normalized prior to being used as training data.

Process 300 may then proceed to act 316, where the patient medical information associated with the data fields defined in act 314 may be used to train the risk model (e.g., a machine learning model) for the patient groups defined in act 312. At least some of the patients (e.g., 10%, 20%, 30%, etc.) in a first patient group (e.g., the PCCS patient group) and a second patient group (e.g., the no PCCS patient group) may be held out by not being used for training the model. The data for the held out patients may be used to obtain a performance score (e.g., sensitivity, specificity) of the trained model. Process 300 may then proceed to act 320, where the trained model may be output. For example, the trained model may be stored for later use in assessing risk of PCCS for new patients not included in the training data set or the held out data set. An example of using a trained model to determine a patient classification associated with developing PCCS is described herein in connection with process 100 of FIG. 1.

As described herein, the inventors have recognized that it may be beneficial to update (e.g., retrain) the model used to assess risk of developing PCCS. For example, if some of the criteria used to initially define the patient groups is unavailable in a new patient dataset, the PCCS criteria may be refined to define the patient groups used to retrain the model. Similarly, different data fields may need to be defined in act 314 for particular hospital systems or different electronic medical record types or formats. Additionally, it may be beneficial to tune the model for a particular hospital system to improve the predictive ability of the model for that particular hospital system. For example, the hospital system may perform a large number (e.g., hundreds, thousands, etc.) of cardiac surgeries over the course of a year, and it may be advantageous to use the patient data from the large number of cardiac surgery patients to tune the risk model to be specific for the patient population of the hospital system.

Returning to process 300 shown in FIG. 3, it may be determined in act 322 whether the risk model is to be updated. If it is determined in act 322 that the model does not need to be updated, process 300 may end. Otherwise, process 300 may proceed to act 324, where it may be determined whether the PCCS criteria should be updated. If it is determined in act 324 that the PCCS criteria should be updated (e.g., because some of the PCCS criteria is not available in the patient dataset used for retraining), process 300 may proceed to act 310, where the PCCS criteria may be updated based on the current patient dataset being used to update the model. If it is determined in act 324 that the PCCS criteria does not need to be updated, process 300 may proceed to act 326, where it is determined whether the data fields used for retraining the model should be updated. If it is determined in act 236 to update the data fields used for training, process 300 may proceed to act 314, where the data fields may defined based on the current patient dataset used to update the risk model. If it is determined in act 326 that the data fields do not need updating, process 300 may proceed to act 316, where the model is trained using the current patient dataset selected for retraining the model. As should be appreciated, the risk model may be retrained at any suitable interval with any suitable training dataset and for any suitable patient population such that the prediction capabilities of the risk model align with the patient population for which risk assessment of developing PCCS is desired. For example, if it is desired to assess risk of developing PCCS over time for a particular patient, the risk model may be trained using longitudinal data, which describes values for the defined data fields at multiple points in time.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A method of predicting whether a patient is likely to develop post-cardiotomy cardiogenic shock (PCCS), the method comprising:
receiving medical information for a patient;
extracting one or more features from the received medical information;
providing the one or more features as input to a trained classification model configured to output a risk assessment that the patient is likely to develop PCCS; and
outputting an indication of the risk assessment.

2. The method of claim 1, wherein the medical information for the patient includes one or more of an electronic health record , a laboratory report, a medical procedure report, physician notes, and a medical imaging report.

3. The method of claim 1, wherein the medical information includes structured data and unstructured data.

4. (canceled).

5. The method of claim 1, wherein the one or more features include left ventricle ejection fraction and/or total bilirubin level.

6. The method of claim 1, further comprising:
receiving data indicating whether the patient developed PCCS; and
retraining the trained classification model based, at least in part, on the received data.

7. The method of claim 1, wherein
the risk assessment includes a numerical value, and
outputting an indication of the risk assessment comprises displaying the numerical value and/or information based on the numerical value on a user interface.

8. The method of claim 7, further comprising:
performing based on the numerical value, categorization of the patient into a risk group of a plurality of risk groups, wherein
outputting the indication of the risk assessment comprises outputting an indication of the risk group for the patient.

9. The method of claim 8, wherein
performing categorization of the patient into a risk group comprises determining whether the numerical value is above a threshold value; and
classifying the high risk for PCCS when it is determined that the numerical value is above the threshold value.

10. The method of claim 8, wherein outputting an indication of the risk group for the patient comprises displaying on a user interface a color coded indication of the risk group.

11. The method of claim 1, wherein
the risk assessment is a categorization of the patient into a risk group of a plurality of risk groups, and
outputting the indication of the risk assessment comprises outputting an indication of the risk group for the patient.

12-16. (canceled).

17. The method of claim 1, further comprising:
providing a user interface configured to display values for the one or more features;
receiving user input via the user interface to change one or more of the values for the one or more features;
simulating a risk assessment that the patient is likely to develop PCCS based, at least in part, on the changed one or more values, to generate a simulated risk assessment; and
displaying, on the user interface, the simulated risk assessment.

18. The method of claim 1, wherein outputting an indication of the risk assessment comprises outputting a cumulative score associated with the risk assessment.

19. A method of training a risk model to predict whether a patient is likely to develop post-cardiotomy cardiogenic shock (PCCS), the method comprising:
receiving a dataset of patient medical information;
selecting, from the dataset of patient medical information, training data based on PCCS criteria and defined data fields, wherein the training data includes patient medical information for at least two risk groups of patients;
training the risk model using the selected training data; and
outputting the trained risk model.

20. The method of claim 19, further comprising;
defining a plurality of PCCS criteria; and
generating the at least two risk groups of patients based on the PCCS criteria.

21. The method of claim 19, further comprising:
receiving input via a user interface regarding the data fields to define; and
defining the data fields based, at least in part, on the received input.

22. The method of claim 19, further comprising:
validating the trained model using at least some patient medical information not used to train the model,
wherein outputting the trained risk model comprises outputting the validated trained model.

23. The method of claim 19, further comprising:
receiving an indication to update the trained risk model; and
retraining the risk model in response to receiving the indication to update the trained risk model.

24-26. (canceled).

27. The method of claim 19, further comprising:
receiving additional medical information; and
retraining the trained risk model based on the additional medical information.

28. The method of claim 20, wherein the additional medical information includes medical information for a plurality of patients at a medical facility on which cardiac surgery was performed.

29. A computer-implemented system for predicting whether a patient is likely to develop post-cardiotomy cardiogenic shock (PCCS), the system comprising:
at least one hardware computer processor; and
at least one non-transitory computer readable medium encoded with a plurality of instructions that, when processed by the at least one hardware computer processor perform a method, the method comprising:
extracting one or more features from medical information for the patient;
providing the one or more features as input to a trained classification model configured to output a risk assessment that the patient is likely to develop PCCS; and
outputting an indication of the risk assessment.

30. (canceled).
